(19) United States
(12) Patent Application Publication
(10) Pub. No.: US 2011/0217391 A1
(43) Pub. Date: Sep. 8, 2011

(54) MATRIX METALLOPROTEASE (MMP) PRODUCTION INHIBITOR

(75) Inventors: Taichi MURAGUCHI, Ashigarakami-gun (JP); Toshiyuki HONMA, Ashigarakami-gun (JP)

(73) Assignee: FUJIFILM CORPORATION, Tokyo (JP)

(21) Appl. No.: 13/039,069

(22) Filed: Mar. 2, 2011

(30) Foreign Application Priority Data
Mar. 3, 2010 (JP) 2010-046565

(51) Int. Cl. A61K 8/97; A61Q 19/08

(52) U.S. Cl. 424/725

(57) ABSTRACT

It is an object of the present invention to provide a method for inhibiting the production of matrix metalloprotease (MMP), wherein skin aging is suppressed and a highly stable and safe component from plants is used. The present invention provides a method for inhibiting the production of matrix metalloprotease (MMP), which comprises administering a Salacia plant extract to a subject in need of inhibition of the production of matrix metalloprotease (MMP).

### TECHNICAL FIELD

The present invention relates to a method for inhibiting the production of matrix metalloprotease (MMP), as well as a matrix metalloprotease (MMP) production inhibitor, a composition comprising the same, and an anti-wrinkle agent. More specifically, the present invention relates to a Method for inhibiting the production of matrix metalloprotease (MMP), as well as a matrix metalloprotease (MMP) production inhibitor which is useful for prevention or improvement of skin aging and which is highly safe, a composition comprising the same, and an anti-wrinkle agent.

### BACKGROUND ART

It has been desired to develop a means for preventing skin change caused by senescence, for physiological and aesthetic skin elements. Mainly, ultraviolet light such as sunlight is largely associated with skin aging. When ultraviolet light is applied to the skin, active oxygen is generated, and the thus generated active oxygen triggers inflammation (Tanpakushitsu bunkai koso I (Protease I), edited by Masanori Tsuru & Masaru Funatsu, 137-169). In particular, with regard to formation of wrinkles, the disappearance of texture, reduction in elasticity and the like, matrix metalloprotease (MMP) is produced by cytokine which is provoked as a result of inflammation, and extracellular matrix components such as collagen and elastin are decreased, thereby causing a structural change. Accumulation of such changes is a cause of formation of wrinkles, the disappearance of texture, and a reduction in elasticity (Y Minami et al., Journal of Nutritional Biochemistry 20, 389-398 (2009); and Han et al., Surgery. 2005 November; 138(5): 932-939).

Matrix metalloprotease (MMP) is a protease, and it is associated with degradation and reconstitution of constituent proteins of extracellular matrixes. At least 11 types of matrix proteases (MMP) have been known with regard to humans. Among them, a total of 8 types, namely, collagenase (two types; MMP-1 and MMP-8), gelatinase (two types; MMP2 and MMP9), stromelysin (three types) and pump-1, have been identified as matrix metalloproteases (MMP), which share structural and functional characteristics but which are clearly different from one another at a gene level. These matrix metalloproteases (MMP) are different from one another in terms of its substrate protein and its expression site. Among such matrix metalloproteases, matrix metalloproteases 2 and 9, which belong to the gelatinase group of MMP, have been known as enzymes which decompose basement membrane components such as type IV collagen and laminin, elastin as a dermal matrix component, and the like. These matrix metalloproteases are produced at a protein level, and the production is triggered by cytokines including TGF-β (a transforming factor) as a typical example, which are formed by irradiation with ultraviolet light (Y. Minami et al., Journal of Nutritional Biochemistry 20, 389-398 (2009); and Han et al., Surgery. 2005 November; 138(5): 932-939). As a result of this reaction, reduction and degeneration of extracellular matrixes, which are important components for skin structure, are provoked, and such reduction and degeneration of extracellular matrixes are considered to become a large factor for formation of wrinides on the skin, etc. (see, for example, Gary J. Fisher et al., Nature, 379(25), 335 (1996); and Gary J. Fisher et al., The New England Journal of Medicine, 337(20), 1419 (1997)). Moreover, in recent studies, matrix metalloprotease 9 has been produced at a high level in rats in which wrinkles have been formed by irradiation with ultraviolet light. The relationship between the production amount of matrix metalloprotease 9 and formation of wrinkles is shown. That is to say, in order to prevent and improve skin aging, it is important to suppress the production of matrix metalloprotease 9 (MMP-9), which is classified into the gelatinase group, on the epidermis directly exposed to ultraviolet light.

In particular, keratinocytes constituting the epidermis susceptible to ultraviolet light exposure and external factors express mainly a large amount of matrix metalloprotease 9. Thus, from the viewpoint of safety and specificity, it has been desired to develop an inhibitor specific to matrix metalloprotease 9. However, at present, EDTA and o-phenanthroline, which block the active center metal of matrix metalloprotease via chelating action, have been experimentally used. These chelating agents are non-specific to matrix metalloprotease, and thus, from the viewpoint of safety, they have been inadequate as external agents for the skin. Furthermore, synthetically produced low-molecular-weight pharmaceutical agents, peptide inhibitors and other agents have also been used. However, in terms of side effects, these agents have also been inadequate as external agents, and particularly, as external agents used in cosmetic products (Bertin et al., Journal of Medicinal Chemistry, 2005, Vol. 48, No. 24). Further, for use in cosmetic products, retinoic acid and ursolic acid have been introduced as MMP9 inhibitors. However, these compounds have high cytotoxicity, and thus, there are concerns about their safety (Lateef et al., American Journal of Pathology, Vol. 165, No. 1, July 2004; and H-J Cha et al., Oncogene (1998) 16, 771-778).

On the other hand, it is said that extracts from the roots and stems of Salacia plants are effective for the prevention or initial treatment of diabetes in the conventional medicine “Ayurveda” in India or Sri Lanka. In recent years, it has been reported that Salacia plants have action to suppress the elevation of blood glucose level, and that the mode of action thereof is caused by action to suppress sugar absorption based on inhibition of α-glucosidase activity (FOOD Style 21, Vol. 6, No. 5, pp. 72-78). However, the action of an extract from Salacia plants on matrix metalloprotease has not yet been studied.

### SUMMARY OF THE INVENTION

It is an object of the present invention to solve the aforementioned problems of the prior art techniques. That is to say, it is an object of the present invention to provide a method for inhibiting the production of matrix metalloprotease (MMP), wherein skin aging is suppressed and a highly stable and safe component from plants is used. It is an object of the present invention to provide a matrix metalloprotease (MMP) production inhibitor which is effective for suppression of skin aging and which comprises, as an active ingredient, a highly stable and safe component from plants.

As a result of intensive studies directed towards achieving the aforementioned object, the present inventors have found that an extract from Salacia plants has action to inhibit the production of matrix metalloprotease 9. Moreover, the present inventors have produced an external agent for the skin comprising such a Salacia plant extract, have applied the produced agent to the skin with reduced tension and elasticity, and have then evaluated the degree of improvement of the skin. As a result, the inventors have demonstrated that the skin condition is improved by the application of the aforementioned external agent for the skin. The present invention has been completed based on these findings.

The present invention provides a method for inhibiting the production of matrix metalloprotease (MMP), which comprises administering a Salacia plant extract to a subject in need of inhibition of the production of matrix metalloprotease (MMP).

The present invention further provides a method for preventing or improving skin aging which comprises administering a Salacia plant extract to a subject in need of prevention or improvement of skin aging.

The present invention further provides a method for suppressing formation of wrinkles, which comprises administering a Salacia plant extract to a subject in need of suppression of formation of wrinkles.

The present invention further provides a matrix metalloprotease (MMP) production inhibitor comprising an extract from Salacia plants as an active ingredient.

The present invention further provides a composition for preventing or improving skin aging, which comprises the matrix metalloprotease (MMP) production inhibitor of the present invention.

The present invention further provides an anti-wrinkle agent comprising an extract from Salacia plants as an active ingredient.

Preferably, the matrix metalloprotease (WIMP) is gelatinase.

Preferably, the matrix metalloprotease (MMP) is matrix metalloprotease-9 (MMP-9).

Preferably, the Salacia plant extract is administered as a composition for preventing or improving skin aging.

Preferably, the skin aging is associated with at least one condition selected from among disintegration of a collagen fiber bundle structure, formation of wrinkles, the disappearance of skin's elasticity, and a reduction in the difference between a sulcus and an elevation on the skin surface.

Preferably, the Salacia plant extract is administered as an external agent for the skin or as a food product.

According to the present invention, there is further provided a use of a Salacia plant extract for the production of a matrix metalloprotease (MMP) production inhibitor.

According to the present invention, there is further provided a use of a Salacia plant extract for the production of anti-wrinkle agent.

### EFFECTS OF THE INVENTION

The method and the matrix metalloprotease production inhibitor of the present invention exhibit effective action to inhibit the production of matrix metalloprotease, and it is useful for suppression of skin aging. In particular, the method and the matrix metalloprotease production inhibitor of the present invention exhibit action to inhibit the production of matrix metalloprotease 9 on the epidermis. An extract from Salacia plants used as an active ingredient in the present invention is derived from plants, and thus, it is highly stable and safe. The matrix metalloprotease production inhibitor of the present invention can be used as a cosmetic product for preventing skin aging caused by senescence and sunlight.

### BRIEF DESCRIPTION OF THE DRAWINGS

FIG. 1 shows the results of a cytotoxicity test.

FIG. 2 shows the results (the expression levels of MMP9 proteins) of a test regarding suppression of the production of matrix metalloprotease 9 on the epidermis.

FIG. 3 shows the results (the expression levels of MMP9 proteins) of a test regarding suppression of the production of matrix metalloprotease 9 on the epidermis.

FIG. 4 shows the results (MMP9 RNA levels) of a test regarding suppression of the production of matrix metalloprotease 9 on the epidermis.

### MODE FOR CARRYING OUT THE INVENTION

Hereinafter, the embodiments of the present invention will be more specifically described.

In the method for inhibiting the production of matrix metalloprotease (MMP) according to the present invention, a Salacia plant extract is administered to a subject in need of inhibition of the production of matrix metalloprotease (MMP). The matrix metalloprotease production inhibitor (hereinafter also referred to as “MMP production inhibitor”) of the present invention comprises an extract from Salacia plants as an active ingredient. The MMP production inhibitor of the present invention inhibits the production of matrix metalloprotease. The present MMP production inhibitor is able to inhibit preferably the production of matrix metalloprotease having gelatinase activity, and particularly preferably, the production of matrix metalloprotease-9.

Salacia plants are plants in the family Celastraceae, which grow wild mainly in Sri Lanka, India and Southeast Asian areas. More specifically, as such a Salacia plant, there is used at least one plant selected from among Salacia reticulata, Salacia oblonga, Salacia prinoides, Salacia chinensis, Salacia latifolia, Salacia burunoniana, Salacia grandiflora, and Salacia macrosperma.

The term “an extract from Salacia plants” is used herein to mean a disintegrated product, a dry product, an extract, dry powders (extract powders) and the like of an edible portion of the Salacia plant, such as a root, a stem, a leaf, a flower or a fruit. One or more types of such portions may be mixed and then used. More preferably, extract powders extracted from roots and stems are used.

The extract powders are prepared by drying a product which is obtained from the above-mentioned edible portion or the like by solvent extraction. An extraction solvent may be selected from among water, alcohols including methanol and ethanol as typical examples, and mixed solvent consisting of water and alcohols or ketones such as acetone. Preferably, water, alcohol or hydrated alcohol is used. More preferably, hot water, ethanol or hydrated ethanol is used as such an extraction solvent. The above-described hydrated alcohol having an alcohol concentration of 30% to 90% by mass, and preferably 40% to 70% by mass, may be used. Examples of the drying method include spray drying and freeze drying, but the examples are not limited thereto.

The MMP production inhibitor of the present invention can be used as a composition for preventing or improving skin aging. That is to say, a composition for preventing or improving skin aging, which comprises the MMP production inhibitor of the present invention, is also included in the scope of the present invention. In addition, according to the present invention, there is provided an anti-wrinkle agent, which comprises a Salacia plant extract as an active ingredient.

In the present invention, the term “skin aging” is interpreted in the broadest sense. For example, the skin aging is associated with at least one condition selected from among disintegration of a collagen fiber bundle structure, formation of wrinkles, the disappearance of skin's elasticity, and a reduction in the difference between a sulcus and an elevation on the skin surface.

In the present invention, when a Salacia plant extract is used as an active ingredient of a matrix metalloprotease 9 production inhibitor in the production of an MMP production inhibitor, a composition for preventing or improving skin aging, which comprises the same, and an anti-wrinkle agent, the extract is preferably mixed in an amount of 0.0005% to 10.0% by mass (hereinafter simply referred to as %) based on the total amount of the composition. If the mixed amount of the Salacia plant extract is less than 0.0005% by mass, the present invention does not have sufficient effects. On the other hand, if the mixed amount exceeds 10.0% by mass, the obtained effects are not comparable to the increased amount, and further, it is not preferable from the viewpoint of coloration or the like.

The state of a Salacia plant extract to be mixed is not particularly limited, and it may be in any state, such as a solid or solution. The Salacia plant extract may be added and mixed at an appropriate time during the production process.

The MMP production inhibitor of the present invention, a composition for preventing or improving skin aging which comprises the same, and an anti-wrinkle agent can be used as external agents for the skin or food products. When they are used as external agents for the skin, they may be used as anti-wrinkle agents, for example. For instance, the MMP production inhibitor of the present invention may be mixed into a skin lotion and may be then used. To such a skin lotion, only the MMP production inhibitor of the present invention may be mixed. Alternatively, the present MMP production inhibitor may be combined with other active ingredients, and the obtained mixture may be then mixed into a skin lotion.

Compounds serving as inhibitors for other matrix metalloproteases and extracts comprising such compounds may be combined as appropriate, and the thus obtained mixture may be then added to the MMP production inhibitor of the present invention, a composition for preventing or improving skin aging which comprises the same, and an anti-wrinkle agent, so as to enhance or reinforce their effects. Specifically, trifluoroacetyl tripeptide-2 (ECM-Protect BG; Ikeda Corporation) serving as a matrix metalloprotease 1 inhibitor, mucopolysaccharide (MDI Complex; Ikeda Corporation) serving as a matrix metalloprotease 2 inhibitor, and the like may be combined and mixed into the aforementioned agents.

To the MMP production inhibitor of the present invention, a composition for preventing or improving skin aging which comprises the same, and an anti-wrinkle agent, the following agents may be mixed, as appropriate, within the range that does not impair the object of the present invention, as well as the above-mentioned agents: moisturizers such as hyaluronic acid, glycerin, polyhydric alcohol and sugar alcohol; color pigments such as a tar pigment and iron oxide; antiseptics such as paraben; anionic surfactants such as fatty acid soap and sodium cetyl sulfate; nonionic surfactants such as polyoxyethylene alkyl ether, polyoxyethylene fatty acid ester, polyoxyethylene polyhydric alcohol fatty acid ester, polyoxyethylene hydrogenated castor oil, polyhydric alcohol fatty acid ester and polyglycerin fatty acid ester; cationic surfactants such as tetraalkylammonium salts; ampholytic surfactants such as betaine type, sulfobetaine type, sulfoamino acid type, and sodium N-stearoyl-L-glutamate; natural surfactants such as lecithin and lysophosphatidylcholine; natural polymers such as gelatin, casein, starch gum Arabic, karaya gum, guar gum, Locust bean gum, gum tragacanth, quince seed, pectin, carrageenan and sodium alginate; semisynthetic polymers such as methyl cellulose, hydroxylethyl cellulose, hydroxylpropyl cellulose, carboxymethylcellulose sodium and ethyl cellulose; synthetic polymers such as polyvinyl alcohol, polyvinyl methyl ether and its copolymer, polyvinylpyrrolidone, sodium polyacrylate, a carboxyvinyl polymer and a polyethylene oxide polymer; thickeners such as xanthan gum; pigments such as titanium oxide; and antioxidants such as dibutylhydroxytoluene.

The dosage form of each of the MMP production inhibitor of the present invention, a composition for preventing or improving skin aging, which comprises the same, and an anti-wrinkle agent, is not particularly limited. Thus, these agents may be used in the form of a bath agent, as well as a cosmetic product generally applied onto the skin. Examples of an applicable dosage form include a commonly used aqueous solution, a W/O type or O/W type emulsion, and powders, such as a granule, and a tablet, which are prepared using a suitable excipient. Specific examples include a liquid agent, a poultice, an embrocation, a gel agent, a cream agent, an emulsion, a pack, a gel, a stick, a sheet, a cataplasm, an aerosol, a lotion, a powder agent, a foaming agent, a skin lotion, a body soap, a soap and a cosmetic agent.

The MMP production inhibitor of the present invention, a composition for preventing or improving skin aging, which comprises the same, and an anti-wrinkle agent, may be administered by either oral administration or parenteral administration. In the case of parenteral administration, percutaneous administration or transmucosal administration may be applied.

The dose of each of the MMP production inhibitor of the present invention, a composition for preventing or improving skin aging, which comprises the same, and an anti-wrinkle agent, may be determined, as appropriate, depending on the type of a Salacia plant extract used as an active ingredient and the amount used, the body weight, skin condition and physical condition of a user, and the like. In general, the active ingredient may be administered in an amount of approximately 1 μg to 50 mg/cm², and preferably approximately 2 μg to 10 mg/cm², for a single administration.

The present invention will be more specifically described in the following examples. However, these examples are not intended to limit the scope of the present invention.

### EXAMPLES

### Production Example 1

0.5 kg of the root and stem portion of Salacia reticulata was crushed. Thereafter, 10 L of water was added to the crushed product, and the obtained mixture was subjected to extraction at 100° C. for 1 hour. The obtained extract was filtrated with a 100-mesh filter, and the filtrate was then subjected to spray drying, so as to obtain 50 g of extract powders. The obtained powders were used as a Salacia plant extract in the following examples.

### Example 1

### Cytotoxicity Test

### (Methods)

Pellicle-derived human normal keratinocytes were inoculated into a 10% (v/v) FBS-containing MEM medium on a 12-well culture plate, at a cell density of 1.9×10⁴ cells/well. Thereafter, the cells were pre-cultured for 24 hours according to an ordinary method. After completion of the pre-culture, the medium was exchanged with a test medium to which an evaluation sample (retinoic acid, ursolic acid, a grape extract, or a Salacia plant extract) had been added, and culture was then carried out for 48 hours. After completion of the culture, the cells were washed with PBS, and a survival test was then carried out according to a common method using MTT. Specifically, a 0.5 mg/mL MTT solution was added to the culture plate, resulting in a concentration of 500 μL/well, and it was then incubated in an incubator for 2 hours. Thereafter, 1.5 mL of dimethyl sulfoxide was added thereto, and the absorbance at a wavelength of 570 nm was then measured. The obtained absorbance was used as an indicator for the number of surviving cells. The number of surviving cells obtained in the case of not adding an evaluation sample was defined as 100, and the cell survival percentage (%) obtained in the case of adding an evaluation sample was obtained.

### (Results)

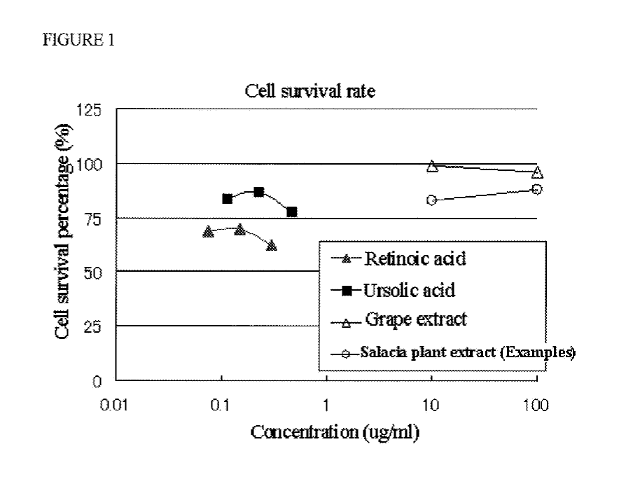

The results are shown in FIG. 1. FIG. 1 shows the cell survival curves when a Salacia plant extract and comparative control compounds and extract were used. As such comparative controls, there were used retinoic acid, and ursolic acid from a sage extract, or a grape extract, the anti-wrinkle effects of which have been reported in publications and the like. When compared with other anti-wrinkle compounds in terms of a cell survival percentage of 60%, the Salacia plant extract of the present invention could be added in a high concentration. As in the case of a grape extract as a mixture, almost no cytotoxicity was observed even in a high concentration range (10 μg/ml or higher) of the Salacia plant extract. That is, it was shown that the Salacia plant extract has high safety to skin cells, and that it can be used over a wide additive concentration range, depending on prescription and situation.

### Example 2

### Test 1 Regarding Suppression of Production of Matrix Metalloprotease 9 on Epidermis

### (Methods)

Pellicle-derived human normal keratinocytes were inoculated into a 10% (v/v) FBS-containing MEM medium on a 12-well culture plate at a cell density of 1.9×10⁴ cells/well. Then, the cells were pre-cultured for 24 hours according to an ordinary method. Thereafter, the medium was exchanged with a test medium which had been prepared by adding TGF (transforming growth factor) β1 or β2 which is known to be released as a matrix metalloprotease 9 production promoting factor from cells upon UV irradiation, to a 1% (v/v) FBS-containing MEM medium to a final concentration of 5 ng/ml, so as to produce a matrix metalloprotease 9 induction test system. Retinoic acid and ursolic acid, each of which had exhibited high cytotoxicity in the evaluation of inhibition, were used in the present test at concentrations which can achieve a cell survival percentage of 70% or more. Specifically, the concentration of retinoic acid was set at 0.15 μg/ml, and the concentration of ursolic acid was set at 0.45 μg/ml. On the other hand, extracts each having low cytotoxicity were used at maximum concentrations, as long as they did not affect the present test. Specifically, the concentration of a grape extract was set at 100 μg/ml, and the concentration of a Salacia plant extract (the present invention) was set at 100 μg/ml. These test substances were each added to a test medium, and the medium was then exchanged with a fresh one. Thereafter, culture was carried out for 48 hours. After completion of the culture, a culture supernatant was recovered. The recovered supernatant was assayed according to the ELISA method with Matrix Metalloprotease-9, Human, Assay (GE Healthcare, RPN2614). Measurement was carried out in accordance with protocols provided by the distributer, and the value at a wavelength of 630 nm was measured using a wavelength-tunable microplate reader, Sunrise Rainbow Thermo (SUNRISE) and was then quantified.

### (Results)

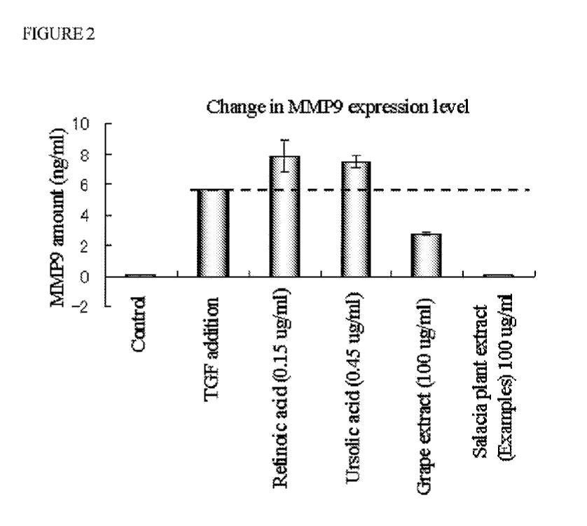

The results are shown in FIG. 2. From the test results shown in FIG. 2, it was found that retinoic acid and ursolic acid, which had been analyzed in publications and the like, did not have the effect of suppressing the production of matrix metalloprotease 9. Moreover, the grape extract exhibited a certain level of suppressive effect, but it could not suppress the expression level to such an extent that it was equivalent to a state in which TGF β1 or β2 had not been added. In contrast, when the Salacia plant extract was added in a concentration of 100 μg/ml, it could suppress the expression level to such an extent that it was almost equivalent to a state in which TGF β1 or β2 had not been added. Thus, the Salacia plant extract exhibited a high effect of suppressing matrix metalloprotease 9.

### Example 3

### Test 2 Regarding Suppression of Production of Matrix Metalloprotease 9 on Epidermis

### (Quantitative Test for Protein Amount and RNA Level)

Pellicle-derived human normal keratinocytes and human normal fibroblasts were each inoculated into a 10% (v/v) FBS-containing MEM medium on a 12-well culture plate at a cell density of 1.9×10⁴ cells/well. Thereafter, the cells were pre-cultured for 24 hours according to an ordinary method. Thereafter, the medium was exchanged with a test medium which had been prepared by adding TGF (transforming growth factor) β1 or β2 which is known to be released as a matrix metalloprotease 9 production promoting factor from cells upon UV irradiation, to a 1% (v/v) FBS-containing MEM medium to a final concentration of 5 ng/ml, so as to produce a matrix metalloprotease 9 induction test system. A Salacia plant extract was added in a concentration of 50 ug/ml to the test medium, and it was then cultured for 32 hours. In addition, as comparative controls, a medium to which the aforementioned TGF had not been added, and a medium to which only the Salacia plant extract had been added, were prepared and examined. After the culture had been carried out for 32 hours, the cells were recovered, and RNA in the cells was then purified using FastPure RNA Kit (Takara Bio Inc.). Thereafter, 1 ug of total RNA was subjected to reverse transcription using PrimeScript 2 High Fidelity RT-PCR Kit (Takara Bio Inc.) The obtained cDNA was quantified by real-time PCR (Stratagene; MX3000) using SYBER Premix Ex Taq (Takara Bio Inc.). Primers purchased and used for matrix metalloprotease 9 were HA133269-F and HA133269-R. As controls, β-actin HA067803-F and HA067803-R were also purchased and used (Takara Bio Inc.). Experimental procedures and analyses were carried out in accordance with instructions included in the kits. Furthermore, the expression levels of the MMP9 proteins 32 hours after the culture were quantified in the same manner as that in Example 2.

### (Results)

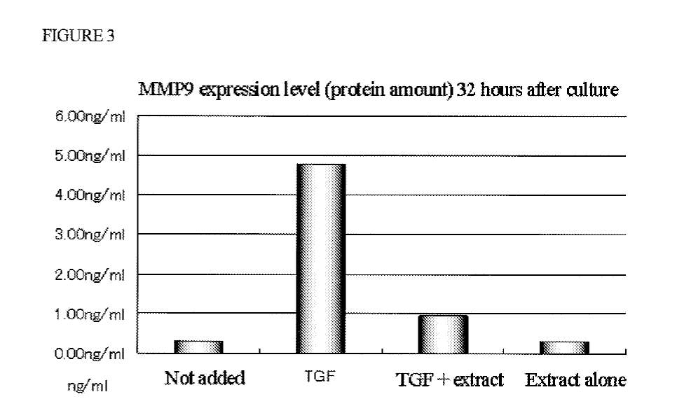

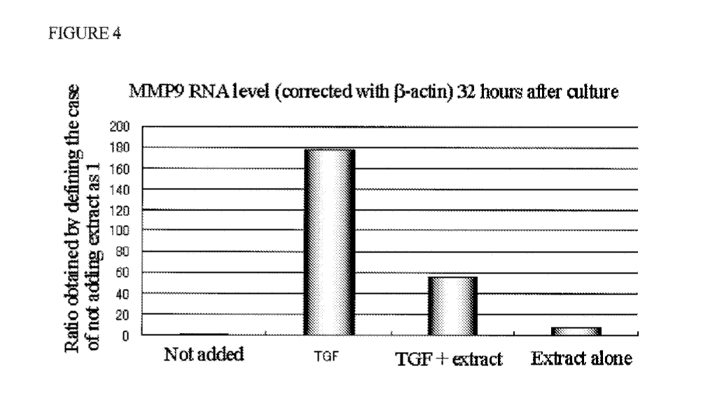

The expression levels of the MMP9 proteins 32 hours after the culture are shown in FIG. 3. The RNA levels of MMP9 (corrected with (3-actin) 32 hours after the culture are shown in FIG. 4. Matrix metalloprotease 9 was significantly increased in keratinocytes, and such increase was suppressed by the Salacia plant extract. This is a phenomenon occurring at an RNA level. This test demonstrated that the above-described effect of suppressing matrix metalloprotease 9 is neither acceleration of a protein degradation system (by stimulation with TGF in this test), nor suppression of extracellular secretion, and that it is the effect of inhibiting the production of matrix metalloprotease 9.

### Example 4

### Wrinkle Improvement Test

In accordance with the prescription shown in Table 1 below, external agents for the skin were produced. Specifically, Ingredient A was added to Ingredient F at room temperature. Also, Ingredient E was dissolved in Ingredient B, and the obtained solution was then added to the above mixed solution. While stirring the obtained mixed solution, Ingredient C was gradually added thereto, to obtain a liquid having viscosity. Finally, Ingredient D was added to the liquid, so as to produce a homogeneous beauty essence 1. At the same time, a placebo beauty essence was produced by the same operations as described above, with the exception that the liquid D was substituted with water.

TABLE 1

| | Ingredient name | Part by mass |
|---|---|---|
| A | Xanthan gum | 0.4% |
| | Hydroxy ethyl cellulose | 0.1% |
| | Carbomer | 0.1% |
| | 1,3-butylene glycol | 5.0% |
| B | Ethanol | 3.0% |
| C | 1% potassium hydroxide aqueous solution | 2.5% |
| D | Salacia plant extract | 1.0% |
| E | Antiseptic (phenoxyethanol) | 0.2% |
| F | Water | adjusted to 100 |

Ten female panelists were selected from 40- to 50-year-old women suffering from decline of the tension and elasticity of the skin, and they were evaluated according to a continuous use test by applying the beauty essence 1 and the placebo beauty essence to them. The 10 panelists were divided into two groups each consisting of 5 panelists, so that their mean age became equivalent in each group (the mean age in beauty essence 1 group: 46.2 years old; the mean age in placebo group: 45.6 years old). In each group, a predetermined amount of essence (approximately 0.6 ml; corresponding to 2 pushes of a quantitative vessel) was administered to a skin portion on which wrinkles had been formed, such as tail of eyes, twice a day (morning and evening), and this administration was continued for 60 days. Forty-eight hours after the final administration, the degree of skin improvement was evaluated on the basis of the following standards: Grade 0: not improved; Grade 1: slightly improved; Grade 2: felt improved; Grade 3: apparently improved; and Grade 4: significantly improved. The results are shown in Table 2. From the results shown in Table 2, it was found that the actual feeling of wrinkle improvement correlates with the action to suppress matrix metalloprotease 9.

TABLE 2

| | Grade 0 | Grade 1 | Grade 2 | Grade 3 | Grade 4 |
|---|---|---|---|---|---|
| Beauty essence 1 | | | 1 | 3 | 1 |
| Placebo beauty essence | 1 | 2 | 1 | 1 | |

1. A method for inhibiting the production of matrix metalloprotease (MMP), which comprises administering a Salacia plant extract to a subject in need of inhibition of the production of matrix metalloprotease (MMP).

2. The method according to claim 1, wherein the matrix metalloprotease (MMP) is gelatinase.

3. The method according to claim 1, wherein the matrix metalloprotease (MMP) is matrix metalloprotease-9 (MMP-9).

4. The method according to claim 1, wherein the Salacia plant extract is administered as a composition for preventing or improving skin aging.

5. The method according to claim 4, wherein the skin aging is associated with at least one condition selected from among disintegration of a collagen fiber bundle structure, formation of wrinkles, the disappearance of skin's elasticity, and a reduction in the difference between a sulcus and an elevation on the skin surface.

6. The method according to claim 1, wherein the Salacia plant extract is administered as an external agent for the skin or as a food product.

7. A method for preventing or improving skin aging which comprises administering a Salacia plant extract to a subject in need of prevention or improvement of skin aging.

8. The method according to claim 7, wherein the skin aging is associated with at least one condition selected from among disintegration of a collagen fiber bundle structure, formation of wrinkles, the disappearance of skin's elasticity, and a reduction in the difference between a sulcus and an elevation on the skin surface.

9. The method according to claim 7, wherein the Salacia plant extract is administered as an external agent for the skin or as a food product.

10. A method for suppressing formation of wrinkles, which comprises administering a Salacia plant extract to a subject in need of suppression of formation of wrinkles.

11. The method according to claim 10, wherein the Salacia plant extract is administered as an external agent for the skin or as a food product.

12. A matrix metalloprotease (MMP) production inhibitor comprising an extract from Salacia plants as an active ingredient.

13. The matrix metalloprotease (MMP) production inhibitor according to claim 12, wherein the matrix metalloprotease (MMP) is gelatinase.

14. The matrix metalloprotease (MMP) production inhibitor according to claim 12, wherein the matrix metalloprotease (MMP) is matrix metalloprotease-9 (MMP-9).

15. The matrix metalloprotease (MMP) production inhibitor according to claim 12, which is used as a composition for preventing or improving skin aging.

16. The matrix metalloprotease (MMP) production inhibitor according to claim 15, wherein the skin aging is associated with at least one condition selected from among disintegration of a collagen fiber bundle structure, formation of wrinkles, the disappearance of skin's elasticity, and a reduction in the difference between a sulcus and an elevation on the skin surface.

17. The matrix metalloprotease (MMP) production inhibitor according to claim 12, which is used as an external agent for the skin or as a food product.

18. A composition for preventing or improving skin aging, which comprises the matrix metalloprotease (MMP) production inhibitor according to claim 12.

19. The composition for preventing or improving skin aging according to claim 18, wherein the skin aging is associated with at least one condition selected from among disintegration of a collagen fiber bundle structure, formation of wrinkles, the disappearance of skin's elasticity, and a reduction in the difference between a sulcus and an elevation on the skin surface.

20. The composition for preventing or improving skin aging according to claim 18, which is used as an external agent for the skin or as a food product.

21. An anti-wrinkle agent comprising an extract from Salacia plants as an active ingredient.

22. The anti-wrinkle agent according to claim 21, which is used as an external agent for the skin or as a food product.
